(19) United States
(12) Patent Application Publication
(10) Pub. No.: US 2018/0140810 A1
(43) Pub. Date: May 24, 2018

(54) CATHETER ASSEMBLIES

(71) Applicant: University of Massachusetts, Boston, MA (US)

(72) Inventor: Oguz Cataltepe, Weston, MA (US)

(73) Assignee: University of Massachusetts, Boston, MA (US)

(21) Appl. No.: 15/578,994

(22) PCT Filed: Jun. 1, 2016
(86) PCT No.: PCT/US16/35211

§ 371 Date: Dec. 1, 2017

Related U.S. Application Data
(60) Provisional application No. 62/169,186, filed on Jun. 1, 2015.

(51) Int. Cl. A61M 27/00

(52) U.S. Cl. CPC A61M 27/006; A61M 2210/0693; A61M 2210/0687

(57) ABSTRACT

A catheter assembly for transferring fluid into and out of a ventricle in the brain is disclosed. The catheter assembly include a sheath permanently installed in the brain, a catheter removably disposed in the sheath, and a connector. In some embodiments, the connector includes inner and outer connection portions, the inner connection portion connected to the sheath and catheter, respectively. A distal end of the catheter may include one or more lumens and/or one or more openings for transferring fluid. The catheter assembly may be connected to a shunt valve and distal (e.g., abdominal) catheter. The distal catheter may be removably disposed within a distal sheath that is permanently attached below the skin of the patient.

### CROSS-REFERENCE TO RELATED APPLICATIONS

This application claims the benefit under 35 U.S.C. § 119(e) to U.S. Provisional Application Ser. No. 62/169,186, entitled “VENTRICULAR SHUT CATHETER ASSEMBLY AND METHOD OF VENTRICULAR CATHETER REPLACEMENT,” filed on Jun. 1, 2015, which is herein incorporated by reference in its entirety.

### FIELD

The invention relates generally to medical catheters, and particularly to catheter assemblies suitable for use in the treatment of hydrocephalus.

### BACKGROUND

Hydrocephalus is a common disorder that is associated with enlarged ventricles in the brain and can be experienced by individuals of all ages. Typically, treatment of hydrocephalus involves placing a shunt catheter, such as a ventriculo-peritoneal shunt (VP shunt) system in the brain. Ventriculo-atrial and ventriculo-pleural shut placements also may be performed in some cases.

### SUMMARY

According to one embodiment, a catheter assembly includes a sheath arranged to be permanently attached to a portion of a brain, a catheter slidably disposed in the sheath, and a connector connected to the sheath and catheter, the connector having one or more ports to allow fluid to be transferred into and out of the brain.

According to another embodiment, a method of installing a catheter assembly in a brain of a patient is disclosed. The method includes installing a sheath into the brain of the patient to create a permanent passageway between a surface of the brain and a ventricle within the brain, and removably inserting a catheter into the ventricle via the sheath.

According to another embodiment, a shunt is disclosed. The shunt includes a catheter assembly arranged to be installed in a patient's brain, a shunt valve, and a distal catheter assembly, the distal catheter assembly including a distal catheter sheath and a distal catheter slidingly disposed in the distal catheter sheath. The distal catheter sheath is arranged to be permanently subcutaneously attached to at least one of a neck, a chest and an abdomen of the patient.

According to still another embodiment, a shunt includes a catheter assembly including a sheath arranged to be permanently attached to a portion of a brain and a catheter slidably disposed in the sheath, a shunt valve, and a distal catheter.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention

In the drawings, each identical or nearly identical component that is illustrated in various figures is represented by a like numeral. For purposes of clarity, not every component may be labeled in every drawing. In the drawings:

FIGS. 1 and 2 are illustrations of known catheters used to treat hydrocephalus;

FIG. 3 is a catheter assembly according to one embodiment;

FIG. 4 is a catheter assembly according to another embodiment;

FIG. 5A is a catheter assembly according to another embodiment;

FIG. 5B is a perspective view of a catheter of the catheter assembly of FIG. 5A;

FIG. 6 is a cross sectional view of an inner connection portion attached to a sheath according to one embodiment;

FIG. 7 is a perspective view of an outer connection portion;

FIG. 8 is a perspective view of a sheath according to one embodiment;

FIG. 9 is a perspective view of a catheter according to one embodiment;

FIG. 10 is a perspective view of a distal end of a catheter according to one embodiment;

FIGS. 11A and 11B are perspective views of a distal end of a catheter according to various embodiments;

FIG. 12 is a schematic representation of an installation and/or removal of a catheter assembly;

FIG. 13 is a schematic representation of an installed catheter assembly according to one embodiment;

FIG. 14A is a schematic representation of an installed catheter assembly and distal catheter assembly according to another embodiment;

FIG. 14B is an enlarged view of the schematic representation of the distal catheter assembly shown in the box labeled 14B in FIG. 14A;

FIG. 15 is a flow chart of a sequence of installing a catheter assembly according to one embodiment; and

FIG. 16 is a flow chart of a sequence of removing and replacing an obstructed catheter of a catheter assembly according to one embodiment.

### DETAILED DESCRIPTION

Hydrocephalus is a disorder that is associated with enlarged ventricles, the fluid chambers located in the center of the brain. As is known, there are four ventricles—the right and left lateral ventricles, the 3^{rd} ventricle and 4^{th} ventricle—within which the choroid plexus produces cerebral spinal fluid (CSF), approximately 300-500 cc of CSF per day. CSF flows through a series of openings or foramens in the brain and out into the subarachnoid space where it is reabsorbed by the venous system. If the CSF pathways become obstructed or obliterated by developmental or acquired abnormalities, CSF accumulates under pressure within the ventricular system. Such accumulation causes the ventricles to begin to dilate, which causes thinning and stretching of the cerebral mantle.

As will be appreciated, a patient may become symptomatic when the ventricles become enlarged and cause increased pressure and/or stretched fibers in surrounding brain tissue. Although the brain can accommodate ventricular dilation to a certain extend without significant neuronal damage, as this process continues, irreversible brain damage may eventually occur.

The most common treatment for hydrocephalus is the placement of ventriculo-peritoneal shunt (VP shunt) systems. Ventriculo-atrial and ventriculo-pleural shut placements also may be performed in some cases. Typically, VP shunts have three main components: a ventricular catheter, a shunt valve and a distal catheter. The ventricular and distal catheter are typically thin, single lumen tubes with multiple holes at the tip.

VP shunts are typically installed via a surgical procedure in which a small incision is made in the scalp, followed by a small drill hole in the skull to open the dura or outer brain membrane. A ventricular catheter may be then inserted into the lateral ventricle. Because ventricles are located in the center of the brain, an inserted ventricular catheter passes through the brain tissue and only the tip of the ventricular catheter is placed into the ventricle. A shunt valve is thereafter attached to the ventricular catheter to control the pressure resulting from enlarged ventricles. That is, the shunt valve may be opened when intraventricular pressure levels exceed a shunt valve opening pressure so that CSF may be drained at times when the pressure within the brain is found to be elevated (e.g., above a threshold pressure). The shunt valve also may be closed to prevent draining CSF when the pressure is appropriate. A distal (e.g., peritoneal, atrial, or pleural) catheter may be connected to the shunt valve for draining the CSF into the abdominal, atrial, or pleural cavity.

Unfortunately, it is very common for patients with VP shunt systems to undergo multiple surgeries for shunt revision because of a malfunction with or failure in the VP shunt system, which causes the patient to again become symptomatic of hydrocephalus. For example, the frequency of shunt malfunction has been reported to be in the range of 40-60% within the first 5 years of implantation. Obstruction of the ventricular catheter is one of the most common reasons for shunt revision surgeries and accounts up to 75% of all cases of revisions. For example, an obstruction of one or more of the perforation holes in the lumen of the catheter with choroid plexus, brain tissue, a clot, debris, and the like, may occur. Patients also may need to undergo a revision as a result of anatomical changes. For example, as a patient grows (e.g., from a newborn to an adolescent), the size of the head or length of the patient may increase and the installed catheter may become too small and, thus, may move outside of the ventricle or the abdomen.

Although shunt revisions are one of the most commonly performed surgeries in neurosurgery, there are numerous problems that can occur during the surgery. Applicant has realized that such problems may be attributable to the current design and technology regarding ventricular catheters. This may include, for example, frequent repeated obstruction of the catheters. As another example, repeat revisions may require re-puncturing of the brain to place a new ventricular catheter, which risks repeatedly injuring the brain tissue. There also may be some difficulty in placing a new ventricular catheter into small ventricles during the surgery. As such, multiple passes may be needed in these cases, which also may expose the patient to potential brain injury. Additionally, the catheter might be placed suboptimally or outside the ventricle when the ventricles are small. There is also a risk of bleeding and brain injury during the surgery, especially in situations where choroid plexus is sucked in the catheter lumen, the choroid plexus having high vascularity.

Because of these challenges in hydrocephalic patients with small, slit-like ventricles, additional tools and techniques such as neuronavigation, endoscopy, or stereotactic implantation may be needed to enhance the accuracy of ventricular catheter placement and to reduce the rate of ventricular catheter re-obstruction. These tools and techniques, however, significantly prolong the duration of surgery and increase the risk for shunt infection, as well as increasing the cost of surgical intervention.

In view of the above, Applicant has realized that by providing a catheter system that is at least partially permanently implanted in the brain during an initial VP shunt surgery, various advantages may be achieved. To that end, embodiments disclosed herein include a catheter assembly having an sheath that is permanently installed in the brain, with a catheter slidably received in the outer sheath for insertion into the ventricle.

As will be appreciated, by having a permanently installed sheath, the brain need not be punctured during each revision surgery. That is, after the sheath is installed, a physician may simply remove and replace the obstructed catheter during a revision procedure. In such embodiments, the new catheter is placed into the same location in the same ventricle through the permanent passageway created via the sheath. This not only may decrease the risk to brain injury that typically accompanies revision surgeries, such as via repeated puncturing, it also may decrease the risk of bleeding. Such a sheath also may ensure that a passageway to the ventricles always remains open. As such, a physician need not worry whether it will be difficult to place the catheter (e.g., if the ventricle is small) or that the replacement of the catheter will be suboptimal or outside the ventricle. In addition, it is expected that the duration of the revision surgery will be shorter than the standard time for revision surgery, because locating the path is much easier since there is already an installed channel to the ventricle. As will be appreciated, a shorter surgical session is advantageous for the patient. In sum, the disclosed catheter assembly may reduce the complexity and invasiveness of revision surgeries and, thus, may make for an improved patient recovery.

Such a permanent passageway to the ventricle also may allow a physician to drain fluid without having an installed catheter. For example, a physician may drain fluid from the ventricles prior to placement of a new catheter during a revision surgery. The passageway also may allow the physician to conduct an exploration of the ventricles with an endoscope, to fenestrate a membrane, to see the location of the choroid plexus or to determine the appropriate length of the new catheter. For example, the physician may insert an endoscope into the passageway to explore the ventricle prior to placement of a new catheter. As will be appreciated, such steps may be taken without further trauma (e.g., puncture) to the brain.

Additionally, unlike catheters that may become too small and slip out as a patient grows, which may necessitate a revision surgery, Applicant has realized that the disclosed sheath may be installed in a newborn patient and may maintain the permanent passageway into the ventricle, even with growth and age. In other words, the sheath may remain installed in the same patient from infancy to adulthood, with revision surgeries only needed to replace the catheter.

Although the disclosed system may significantly decrease trauma to the brain during revision surgeries, Applicant has realized that some surgeons may still prefer to use existing catheter systems. For example, the sheath may create a slightly larger cross sectional opening in the brain as compared to existing catheter, which may be undesirable. While the catheter assembly may be designed such that diameter of the sheath is the same as the diameter of existing catheters, Applicant believes that the improved surgical experience of the disclosed catheter assembly, even with a larger cross-sectional opening in the brain, will encourage surgeons to not only consider using the assembly but to also adopt the disclosed cather assembly as their preferred treatment option.

According to another aspect, the catheter assembly may be designed to reduce the occurrence of catheter obstructions. For example, while existing ventricular catheters may be used in the assembly, the assembly also may include a newly designed catheter. Such a newly designed catheter may include one or more side-openings in the catheter wall at the ventricular (e.g., distal) end, which may include a slit or fenestration hole having any desired shape such as an elongated opening, a slit opening, an eye-drop shaped opening, circular holes, or other suitable shapes or combinations of shapes. Such newly designed catheters also may include one or more lumens (e.g., 3-4 lumens) located at a distal end, the one or more lumens being in communication with a central lumen extending along a remainder of the length of the catheter. As will be appreciated, in such embodiments, if one of the lumens becomes obstructed, such as via a clot, CSF may still pass through one or more of the other lumens at the distal end of the catheter to the central catheter lumen.

According to still another aspect, a distal catheter assembly that is connected to the shunt valve for draining the CSF into the abdominal, atrial, or pleural cavity is disclosed. As is known, traditional catheters used to drain CSF include a tube with a single lumen and openings at the tip and/or side. Such catheters typically extends from the brain to the abdominal cavity and are positioned subcutaneously in the neck, chest and/or abdominal wall. Without wishing to be bound by theory, these catheters may become calcified over time, may become attached to the subcutaneous tissue, may need to be replaced when they become broken, obstructed and/or shortened. Traditionally, surgery to remove an old distal catheter includes multiple incisions on the head, neck, chest and/or abdomen to remove old catheter pieces (e.g., piece by piece). Alternatively, the old, broken distal catheter may be left in place, with a new catheter being inserted subcutaneously to form a new, second tract. In other words, the patient would live with the old and new catheters under his or her skin.

To that end, and similar to the catheter assembly, Applicant has realized that by providing a distal catheter assembly that is at least partially permanently implanted in the neck, chest, and/or abdomen, advantages may be realized. As such, embodiments disclosed herein comprises a VP shut with a catheter assembly attached to the brain, a shunt valve and a distal catheter assembly that connects the shunt valve to the neck, chest and/or abdomen. In some embodiments, the distal catheter assembly includes a distal sheath and a distal catheter that is slidably received in the distal sheath. Such a distal catheter may be placed under the skin with contact between the head and abdominal incision via a shut tunneler In use, to replace the catheter, the physician need only remove the distal catheter, while the distal sheath remains attached to the body.

As will be appreciated, having such a permanent subcutaneous path (e.g., the distal sheath) from the shunt valve located at the head to abdominal cavity, may afford an opportunity to remove/replace abdominal catheters through a small incision at the head. In such an embodiment, a new abdominal catheter may be placed through this incision without having to open an abdominal incision and without re-entering the abdominal cavity.

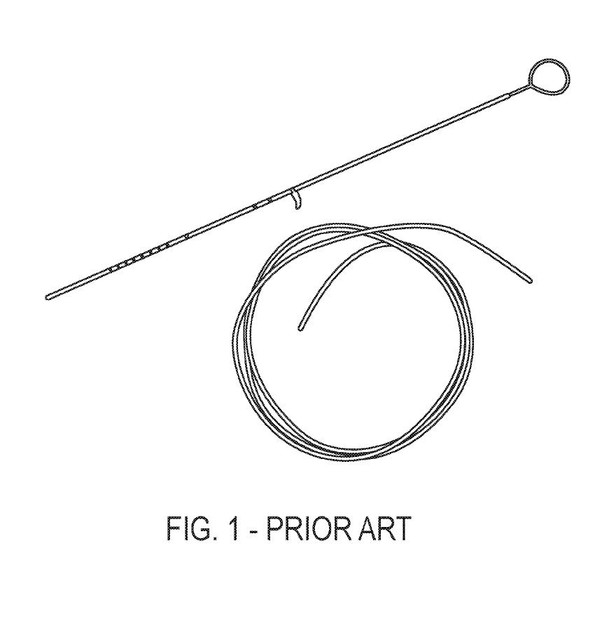

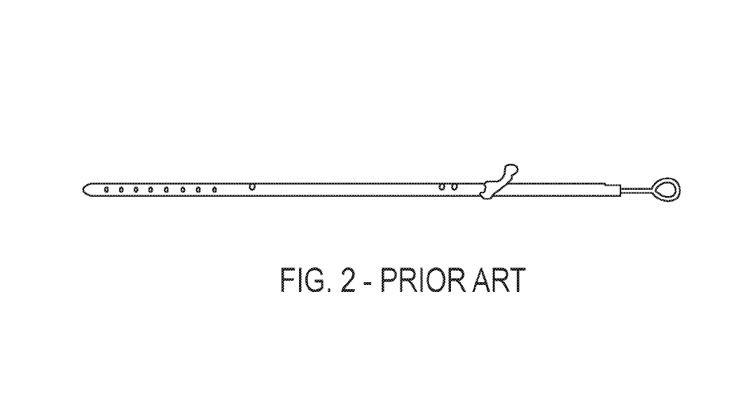

Turning now to the figures, FIGS. 1 and 2 illustrate existing prior art ventricular catheters. As is known, such catheters include thin, tubular, tubes (e.g., silicone elastomer tubes), with a single lumen extending along the entire length and various numbers of perforations at the distal end. As will be appreciated, the number of holes may vary depending on the model, such as 16, 20, or 32 holes. Generally, the holes are arranged in 4 or 6 rows of opposing placed perforations, with a total perforated segment of 10-15 mm length at the most end of the catheter.

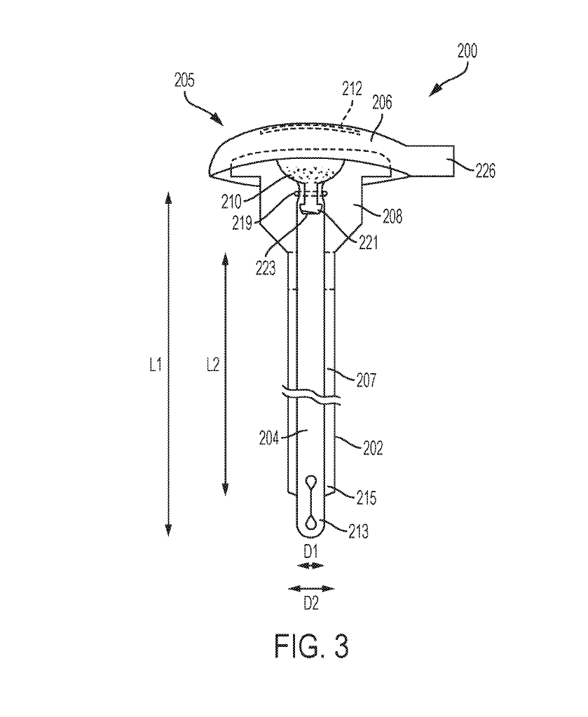

FIG. 3 includes a cross sectional schematic representation of a catheter assembly 200 according to the present disclosure. As will be appreciated, such a catheter assembly may be used as a ventricular catheter assembly in ventriculo-peritoneal (VP) shunts. For purposes herein, a ventricular catheter assembly includes a catheter assembly that is located within or in communication with a ventricle of a brain. Such a ventricular catheter assembly may be used with the other components of the VP shunt—a shunt valve and a distal catheter. As will be further appreciated, the catheter assembly also may be used with ventriculo-atrial and ventriculo-pleural shuts, or with other shunts.

As shown in FIG. 3, in some embodiments, the catheter assembly 200 includes a catheter sheath 202, a catheter 204 slidably received within the sheath, and connector 205 that connects the proximal ends of the sheath 202 and catheter 204 to the shunt valve (not shown). As will be appreciated, the connector 205 may be arranged to allow CSF to pass from the ventricles in the brain, as will be described.

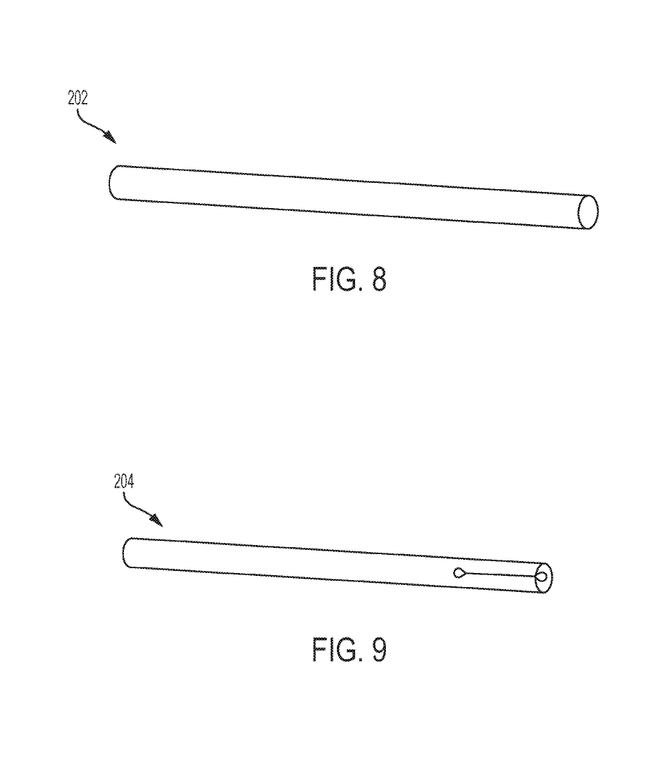

For purposes herein the sheath includes an outer covering that is placed over the catheter. In some embodiments, the sheath may be cylindrical in shape, although it will be appreciated that the sheath may have other shapes. As shown in this FIG. 8, the sheath includes openings at both a top and bottom, the catheter being slidable into each of the openings for positioning a distal end of the catheter into the ventricles of the brain.

Turning back to FIG. 3, in some embodiments, the outer diameter D1 of the catheter 204 is arranged to be less than or nearly equal to the inner diameter D2 of the sheath such that the catheter 204 may be slidably inserted into the sheath. For example, the outer diameter D1 of the catheter 204 may be between about 2 mm and 3 mm, while the inner diameter D2 of the sheath may be between about 2 mm and 3.5 m. In such embodiments, the sheath may have an outer diameter of between about 2.5 mm and 4 mm, such that a wall thickness of the sheath may be between about 0.3 mm and 0.8 mm. The catheter may have an inner diameter of between about 1.5 mm and 2.5 mm, such that a wall thickness of the catheter is between about 0.3 mm and 0.8 mm. In one illustrative embodiment, the inner and outer diameters of the sheath are about 2.5 mm and 3 mm, respectively, with a wall thickness of about 0.5 mm. In such an illustrative embodiment, the catheter may have inner and outer diameters of about 1.5 mm and 2 mm, respectively, with a wall thickness of about 0.5 mm.

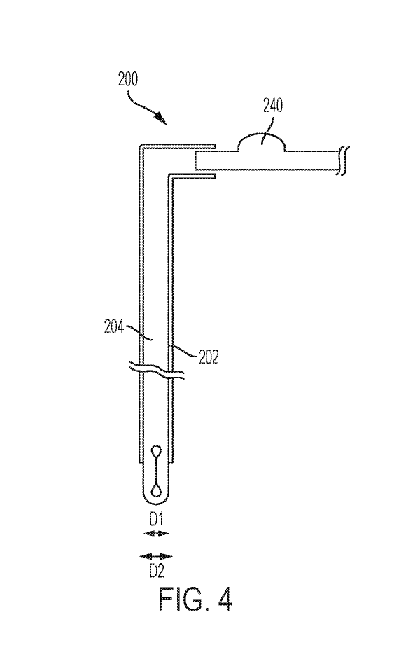

In some embodiments, as illustrated in FIG. 3, the assembly is arranged such that there is a space 207 between the outer surface of the catheter 204 and the inner surface of the sheath 202. As will be appreciated, in such embodiments, the outer diameter D1 of the catheter 204 and/or the inner diameter D2 of the sheath may be larger than in embodiments in which there is no space between the catheter and sheath (see, e.g., FIG. 4). In some embodiments, the space equals a difference between the outer diameter D1 of the catheter 204 and the inner diameter D2 of the sheath, which may be between about 0.25 and 0.5 mm, although other suitable distances may be used. In such embodiments, CSF may be permitted to travel within the space and to the connector, thus allowing additional drainage channel for the CSF. As will be described, the assembly 200 may include a connector 205 that is arranged to connect the catheter 204 and sheath 202 to each other and to the shunt valve (not shown).

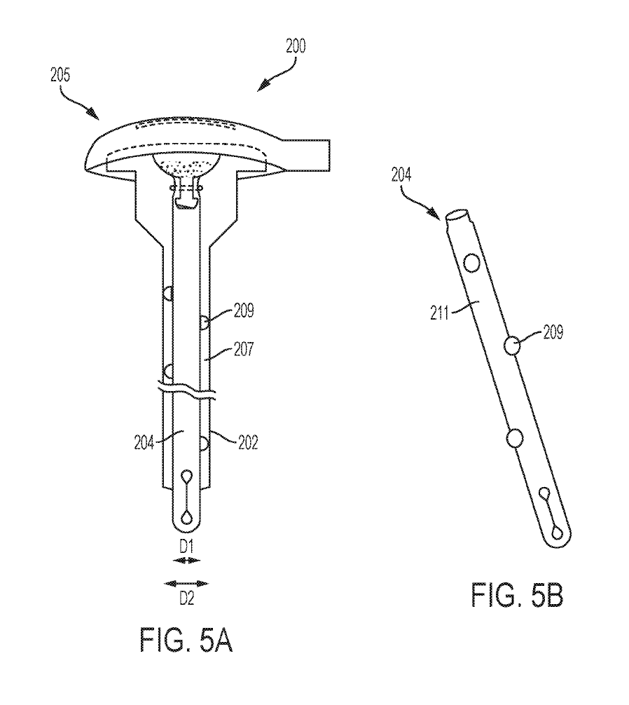

In some embodiments, as shown in FIGS. 5A and 5B, the catheter may include one or more protrusions 209 extending outwardly from the outer surface 211 of the catheter 204 for maintaining the space between the catheter 204 and sheath 202. As will be appreciated, the protrusions 209 may be any shape and be positioned at any suitable location on the catheter 204. As will be further appreciated, although the protrusions are shown on the catheter, in other embodiments, the protrusions may be located on the inside surface of the sheath.

In some embodiments, as shown in FIG. 4, the catheter 204 fits snugly within the sheath such that the outer diameter of the catheter need only be slightly less than the inner diameter of the sheath. As will be appreciated, the difference in diameters need only be large enough so that the catheter may be removable from the sheath during a revision procedure. In this regard, the outer diameter D1 of the catheter 204 may be nearly the same as inner diameter D2 of the sheath. In such embodiments, the catheter and sheath may be connected directly to the shunt valve 240. As will be appreciated, in other embodiments, the sheath and catheter may be connected to the shunt valve via a connector.

Turning back to FIG. 3, in some embodiments, a length L1 of the catheter is longer than a length L2 of the sheath. In such embodiments, a distal end 213 of the catheter 204 may extend outwardly from a distal end 215 of the sheath 202 such that the catheter 204 may be inserted into the ventricle (not shown) for extracting or withdrawing CSF. In such embodiments, the catheter 204 may be longer than the catheter sheath 202 to provide a larger CSF contact surface to side openings at the distal end, such as at the most distal end, of the catheter tip in the ventricle for better fluid drainage. In some embodiments, the catheter may extend outwardly beyond the tip between about 0.5 cm and 2 cm.

In some embodiments, the length of the sheath may be determined based on the patient's head size and age. For example, the sheath length may be between about 2 cm and 10 cm installed. As will be appreciated, the uninstalled sheath may be between about 10 and 20 cm, such that the surgeon may cut the sheath to the needed size.

In some embodiments, the length of the installed catheter is between about 4 cm and 12 cm. As with the sheath, the installed length will depend on the patient's age and head size and may be provided in a length of approximately 12-22 cm, such that the surgeon may cut the catheter to size.

As shown in FIGS. 3 and 6-7, the connector 205 includes outer 206 and inner 208 connection portions for connecting the catheter 204 and sheath 202, respectively, to the valve. As will be appreciated, although the connector is shown as having two connection portions, in other embodiments, the connector may be a single piece. Additionally, although the connector is shown as being removably attached to the catheter and sheath, in other embodiments, one or more of the connection portions may be permanently attached to the respective part. In use, the outer connection portion is joined to the inner connection portion, as shown in FIG. 3. For example, the first connector portion may be press fit, snap fit, slip fit, threaded or otherwise suitably connected to the inner connector portion.

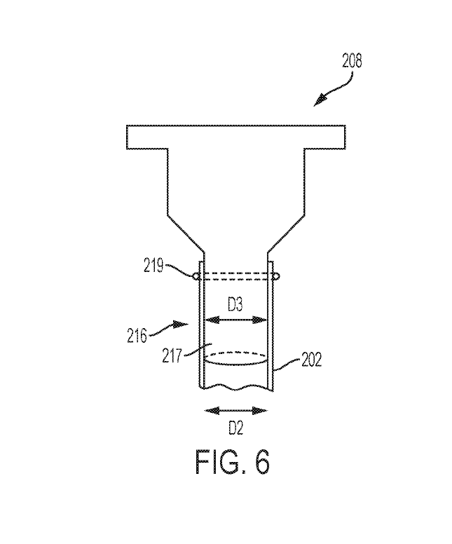

FIG. 6 illustrates the inner connection piece 208, which is arranged to be attached to the sheath 202 according to the present disclosure. As shown in this figure the sheath 202 may attached to inner connection piece 208 via a port 216. In this regard, the wall of the sheath 202 may be slip fit over a distal end 217 of the inner connection portion 208. In some embodiments, the sheath 202 is also secured to the inner connection portion 208 by being tied with a silk tie or thread 219.

In one illustrative embodiment, the outer diameter D3 of the port 216 (e.g., the outer diameter of the distal end 217 of the inner connection portion 208) may be about 3 mm and the inner diameter D2 of the sheath 202 may be about 3 mm, so that a press fit can be achieved. As will be appreciated, other suitable connections may be used to join the sheath and inner connection portion. In other embodiments, the sheath and outer connection portion also may be integrally formed. In one embodiment, a length of the sheath placed over the proximal end of the outer connection portion is about 5 mm.

In some embodiments, the inner connection portion 208 fits a drill hole in the skull of the patient and is attached to the patient. Once installed, the inner portion 208 and the sheath 202 may be permanently installed and, thus, not be removed except under unusual conditions, for example if there is an infection present.

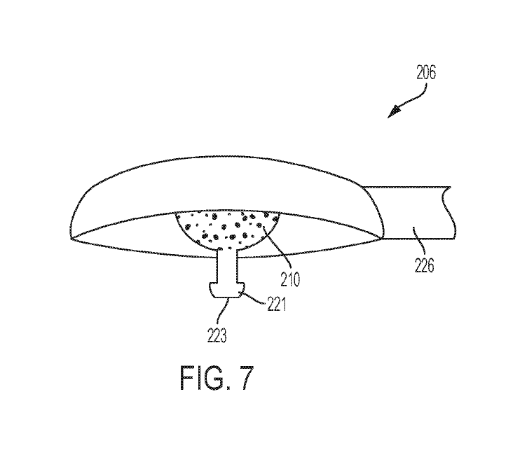

FIGS. 3 and 7 illustrates the outer connection portion 206, which is arranged to be connected to the catheter 204. As with the inner connection portion, the catheter 204 may be slip fit onto a distal end 221 of the outer connection portion 206 and joined to the outer connection portion at port 223. A silk tie or thread 219 also may be tied around the catheter 204 and outer connection portion 206 to secure the catheter to the outer connection portion 206. As will be appreciated, the catheter 202 and outer connection portion 206 may be joined in other suitable methods of may be integrally formed in other embodiments.

In some embodiment, a diameter of the port 223 is greater than the inner diameter of the catheter 204 such that the catheter is unlikely to come loose during use. In one illustrative embodiment, the inner diameter of the catheter 204 is 1.2 mm while the diameter of the port 223 is 1.4 mm. In some embodiments, a length of the catheter placed over the proximal end of the outer connection portion is about 3 mm. In some embodiments, the catheter may be inserted into the ventricle by passing the catheter through the sheath 202, with the outer connection portion 206 attached to the catheter 204. Next, the outer connector portion 206 may be snugly inserted into the inner connection portion 208, thus completing the installation of the shunt.

As also shown in FIGS. 3 and 7, the outer connection portion may have one or more ports for allowing fluid to pass into or out of the connector and, thus, brain. For example, a first port 212 (see FIG. 3), may be located on a top of the outer connection portion 206, which may have self-sealing penetrable dome, such as the type of self-sealing membrane found in vials of injectable medications. In some embodiments, the port 212 may be used to remove CSF (e.g., to perform an analysis of the CSF), to inject a fluid (e.g., to inject a medication) and/or to measure the pressure of the fluid within the ventricular catheter assembly (and thereby the pressure in the ventricle). The outer connection portion 206 also may include a port 226, which may be used to connect the catheter assembly 200 to a shunt valve (not shown) to allow CSF to be withdrawn, as appropriate.

The outer connection portion 206 also may include a plurality of holes or other openings 210 that permit withdrawal of fluid from the space between the sheath 202 and the catheter 204. In some embodiments, the portion of the outer connection portion 206 having the openings 210 may be about 4 mm in diameter and maybe semi-hemispherical in shape. As previously described, the outer connection 206 also may include a port 223 at the distal end 221 for allowing withdrawal of fluid from the catheter 202.

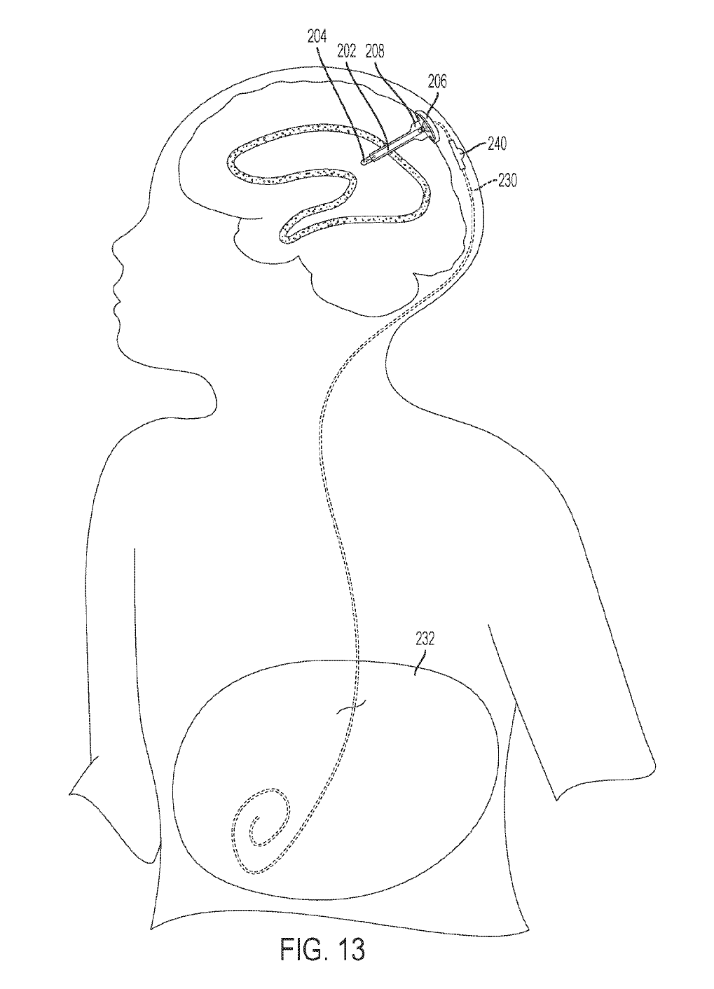

FIG. 8 is an illustration in perspective view of the sheath 202 according to the present disclosure. In the embodiment shown, the sheath 202 a thin, hollow tube. The sheath may be formed of a silicone elastomer, although other suitable materials may be used. The sheath also may be coated with one or more coatings, such as anticoagulants and/or antibiotics. Although the sheath is shown as being cylindrically shaped, it will be appreciated that the sheath may have other suitable shapes. In some embodiments, the sheath includes an obturator (not illustrated) located therein. The catheter sheath 202 and the obturator may be inserted into the ventricle using the standard ventricular catheter placement technique, after which time the obturator may be removed. An illustration of a sheath (and inner connection portion) installed in the brain may be seen in FIG. 12. As will be appreciated, once installed, the hollow sheath 202 creates an access tunnel or passageway between the surface of the brain and a ventricle, which may be used for catheter placement.

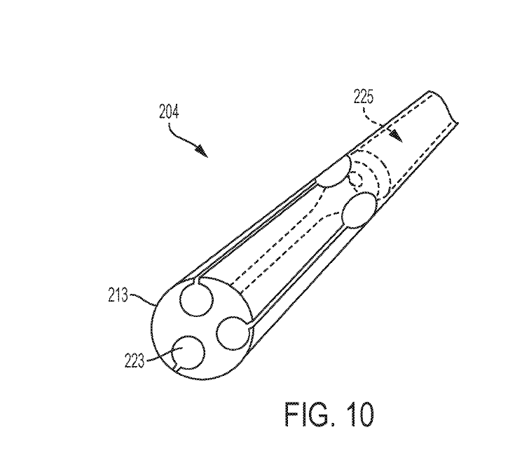

FIG. 9 is a perspective view of a catheter 204 according to the present disclosure. In the embodiment shown, the catheter is a tubular tube, which may be inserted into the sheath (and into the ventricle). As with the sheath, the catheter may be formed of a silicone elastomer, although other suitable materials may be used. The catheter also may be coated with one or more coatings, such as anticoagulants and/or antibiotics. The catheter may be longer than the catheter sheath, so that the distal end of the catheter extends beyond the distal end of the catheter sheath into the ventricle.

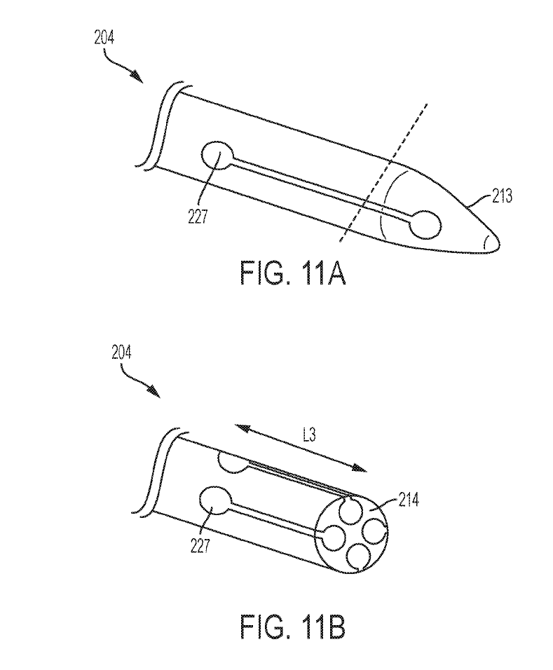

As shown in FIG. 10, in some embodiments, the catheter 204 includes multiple inner channels that are interconnected and communicate with a single lumen. For example, the distal end 213 of the catheter may have one or more lumens 223, which join a central lumen channel 225 extending along the remainder of the length of the catheter 204. In one such example, the catheter may include 3-4 lumens. For purposes herein, the distal end of the catheter having the lumens may include the last 1 to 2 cm of the catheter.

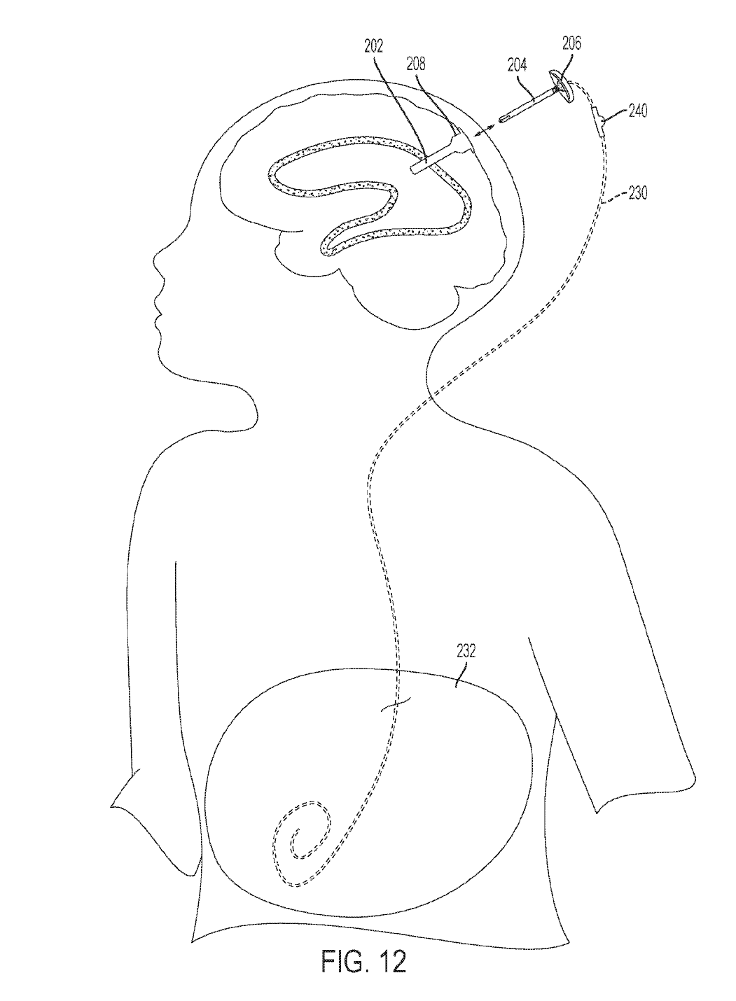

As shown in FIGS. 9 and 11A-B, the distal end 213 of the catheter 204 also may include one or more openings in the catheter wall, such longitudinal slits 227. As will be appreciated, although slits 227 are shown in FIGS. 9 and 11A-B, the openings may have any suitable size or shape. For example, the openings may be an elongated opening, an eye-drop shaped opening, circular holes, or another suitable shapes or combinations of shapes. In some embodiments, the slits may have a length L3 of between about 1 cm and 3 cm. In one illustrative embodiment, the length may be about 2 cm. In some embodiments, a width of the slit may be between about 0.2 mm and 0.8 mm, or about 0.5 mm. As with the lumens, the openings may be placed along the last 1 to 2 cm of the catheter length (e.g., at the distal end) FIG. 11A is an illustration of a perspective view of an embodiment in which the distal end 213 of the catheter has not been cut off. As will be appreciated, the dashed line shown in FIG. 11A indicates where the section shown in FIG. 11B has been removed.

In some embodiments, the catheter 204 allows CSF drainage through wall slits and one or multiple elongated or ellipsoid openings at the tip. Multiple channels can be connected to a wide variety of ellipsoid openings. As will be appreciated, known catheters tiny holes. The ellipsoid openings and slit walls may minimize aspiration of tissues into the catheter lumen, and decrease risk of catheter obstruction.

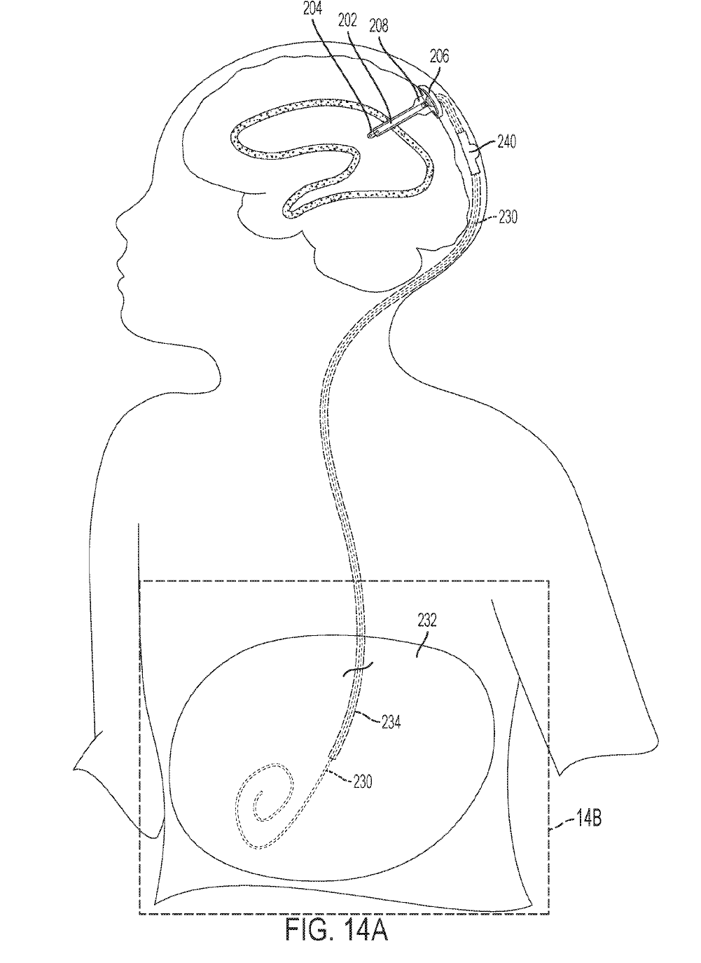

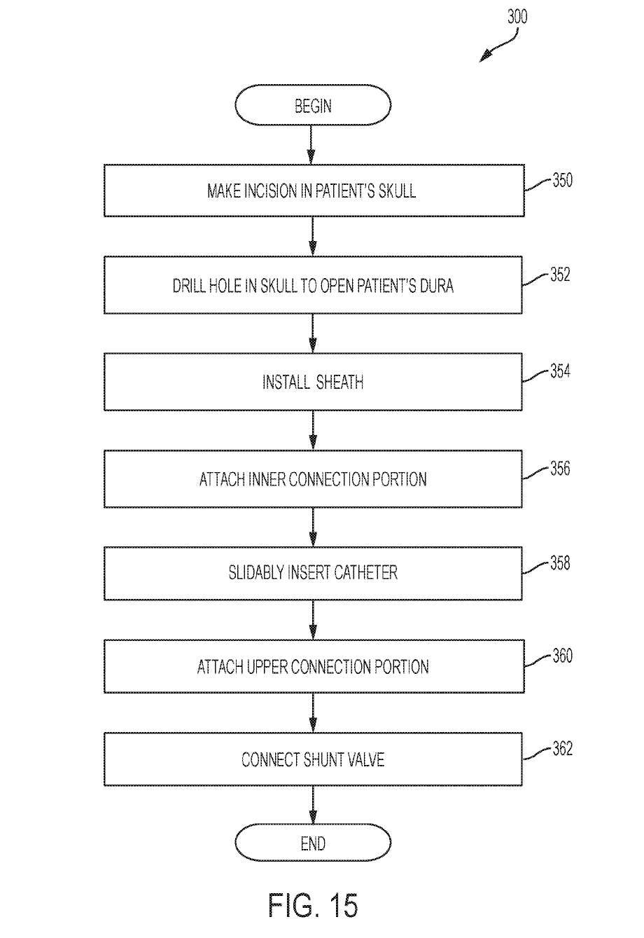

According to another aspect, as illustrated in FIGS. 12 and 13 and the flow chart in FIG. 15, a method of installing the catheter assembly is disclosed. According to one embodiment, the method 300 includes making a small incision on a scalp 350 of a patient and drilling a small hole into the skull of the patient, opening the patient's dura 352. Next, the method includes installing the sheath 354, e.g., with a obturator, into the brain to provide a passageway between a surface of said brain and a ventricle within said brain. If used, the obturator may then be removed. An example of a patient with such an installed sheath is seen in FIG. 12. Next, a proximal end of the sheath is attached to the inner connection portion 356 (e.g., via the port) and the sheath and inner connection portion are permanently attached to the skull. A catheter is removably inserted into the sheath 358 (via the distal end), until the distal end is inserted into the ventricle within said brain. As will be appreciated, the catheter may include an existing catheter or the catheter may include a catheter having a distal end with one or more lumens and/or one or more openings (e.g., slits). As will be further appreciated, the proximal end of the catheter may be attached to an outer connection portion 206, which is thereafter attached to the inner connection portion. A port 226 of the outer connection portion 206 may thereafter be connected to the shunt valve 240 of the VP shunt 362. As will be appreciated, with such an installation, the sheath 202 creates a permanent access tunnel into the ventricle.

In embodiments in which the catheter is snugly fit within the sheath and a connector is not used, as shown in FIG. 4, the method of installing the catheter assembly may be similar to that shown in FIG. 15, except for the steps including the connector. In other words, the method may include making an incision in the patient's scalp 350, drilling a small hole into the patient's skull to open the patient's dura 352, installing the sheath 354, removably inserting the catheter 358, and attaching the catheter and sheath to the shunt valve 362.

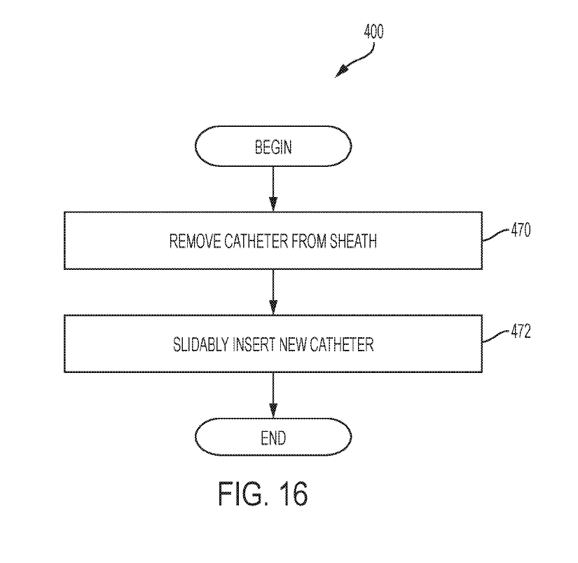

According to another embodiment, a method of removing replacing the catheter in instances of a catheter are disclosed. Such a method 400 is shown in the flow chart of FIG. 16. In such an embodiment, the method may include opening at the head around the shunt valve and disconnecting the sheath-catheter from shunt valve, Next, the method may include removing the obstructed catheter from the sheath 370 and thereafter slidably inserting a new catheter into the sheath 372. The new catheter may have an outer connection portion attached to a distal end, which will thereafter be attached to the inner connection portion in the brain. As will be appreciated, the catheter is inserted until the distal end of the catheter is inserted into the ventricle. As will be further appreciated, during this procedure, only the catheter 204 need be removed and replaced, with the sheath 202 remaining installed in the brain during the revision surgery.

According to another aspect, a distal catheter assembly is disclosed. As shown in FIGS. 12 and 13, for example, a distal catheter 230 may be connected to the shunt valve 240 to transfer the fluid from the brain to the abdominal cavity 232. In some embodiments, as shown in FIG. 12, the distal catheter may include a known catheter having a single lumen and is placed subcutaneously, extending from the brain to the abdominal cavity (e.g., via the neck, chest and abdomen) for draining CSF. As will be appreciated, one of the newly designed catheters disclosed herein also may be used in this configuration.

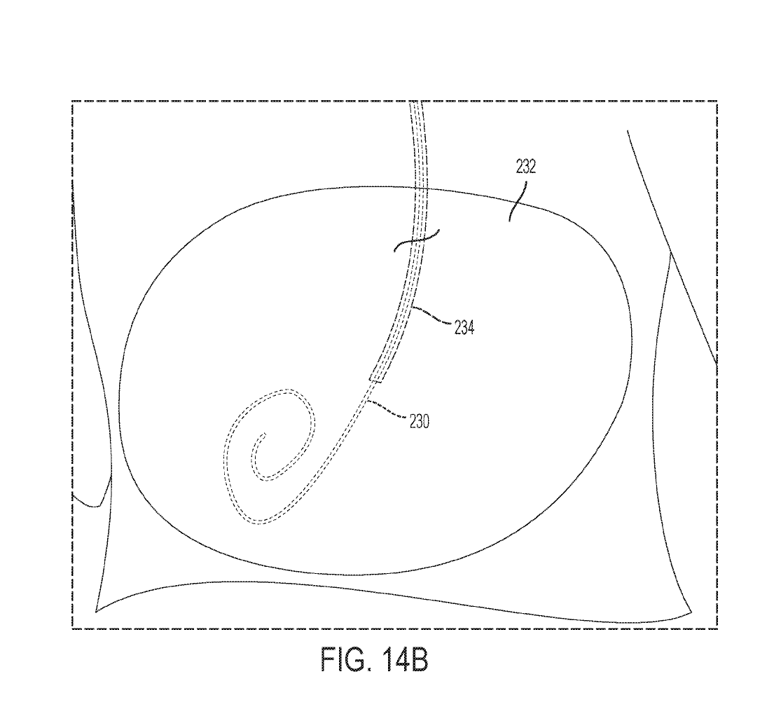

As is known, catheters calcify over time and may break, become obstructed, separate or become shortened. Applicant has realized that by creating a permanent subcutaneous passageway, similar to the permanent passageway created in the brain, advantages may be realized. To that end, and as shown in FIGS. 14A and 14B, in some embodiments, a distal catheter assembly may include a distal sheath 234 extending from the brain to the abdominal cavity 232 and a distal catheter 230 slidably disposed within the distal sheath.

As shown in FIGS. 14A and 14B, in one embodiment, the distal catheter 230 may fit snugly within the distal sheath 234. As with other embodiments, the diameter of the distal sheath in this embodiment need only be slightly larger than the catheter such that the distal catheter may be removed from the distal sheath. As shown in FIG. 14B, a portion of the distal catheter 230 may extend outwardly beyond the distal sheath 234. For example, the length of the distal catheter in the abdominal cavity may be between about 30-40 cm whereas the length of the distal sheath may only be between about 10 to 15 cm long. In such embodiments, the distal catheter may include multiple side holes or slits at the distal end (e.g., over the last 5 cm of the catheter). As also shown in this figure, the distal catheter 230 and distal sheath 234 may be connected to the shunt valve 240.

According to another embodiment, a method of removing and replacing an obstructed distal catheter is disclosed. In such an embodiment, the method may include opening at the head around the shunt valve, disconnecting the sheath and catheter from shunt valve, pulling out the distal catheter without removing the distal sheath and slidably inserting a new abdominal catheter through the distal sheath from the same incision in the head. As will be appreciated, because there is a permanent path from head to the abdominal cavity through the previously placed distal sheath, additional incisions need not be made in the patient.

In some embodiments, the distal catheter may have an outer diameter of between about 2 mm and 3 mm or about 2.5 mm. The inner diameter may be between about 1 mm and 2 mm or about 1.5 mm. As will be appreciated, the wall thickness may be between about 0.3 mm and 0.8 mm or about 0.5 mm. The installed length will also depend on the patient's age and body size and may range between about 60 cm and 90 cm, and may be cut to size from a catheter that is between about 70 and 100 cm in length, or about 90 cm in length.

In some embodiments, the distal sheath may have an outer diameter of between about 2.5 mm to 3.6 mm or about 2.6 mm. The inner diameter may be between about 2.1 mm and 3.1 mm. The wall thickness may be between about 0.3 and 0.6 mm, or about 0.5 mm. The length of the sheath also may be between about 50 cm to 70 cm installed, although this length with vary based on the age and size of the patient. The sheath may be cut to size from a sheath that is between about 70 and 100 cm in length, or about 90 cm in length.

While the ventricular catheter assembly can be used as part of a ventriculo-peritoneal shunt, it can also be used to remove fluid that is then delivered to locations other than the abdomen of the patient, e.g., as part of an external ventricular drainage system.

Also, although the catheter assembly has been disclosed for use in removing fluid in the brain, it will be appreciated, that the catheter assembly may be installed in other location of the body to remove other fluids.

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A catheter assembly, comprising:
a sheath arranged to be permanently attached to a portion of a brain;
a catheter slidably disposed in the sheath; and
a connector connected to the sheath and catheter, the connector having one or more ports to allow fluid to be transferred into and out of the brain.

2. The catheter assembly of claim 1, wherein the connector includes inner and outer connection portions, the inner connection portion being connected to the sheath and the outer connection porting being connected to the catheter.

3. The catheter assembly of claim 2, wherein the inner connection portion is arranged to be attached to the skull of the patient.

4. The catheter assembly of claim 1, wherein the outer connection portion comprises one or more ports for transferring fluid into and out of the brain.

5. The catheter assembly of claim 4, wherein a proximal end of the catheter is attached to one of the one or more ports.

6. The catheter assembly of claim 1, wherein a distal end of the catheter is arranged to be inserted into a ventricle of the brain via the sheath.

7. The catheter assembly of claim 1, wherein the sheath has an inner diameter that is larger than an outer diameter of the catheter.

8. The catheter assembly of claim 1, wherein the catheter has a length that is longer than a length of the sheath.

9. The catheter assembly of claim 1, wherein the catheter fits snugly within the sheath.

10. The catheter assembly of claim 1, further comprising a space between an outer surface of the catheter and an inner surface of the sheath.

11. The catheter assembly of claim 10, wherein the space equals a difference between an outer diameter of the catheter and an inner diameter of the sheath.

12. The catheter assembly of claim 11, wherein the difference between the outer diameter of the catheter and the inner diameter of the sheath is between about 0.25 and 0.5 mm.

13. The catheter assembly of claim 1, wherein a distal end of the catheter comprises two or more lumens in communication with a single lumen extending along a remainder of a length of the catheter.

14. The catheter assembly of claim 1, wherein a distal end of the catheter comprises one or more side openings for transferring fluid into the catheter.

15. The catheter assembly of claim 14, wherein the one or more openings include one or more elongated slits or other shapes.

16. The catheter assembly of claim 14, wherein the one or more side openings are disposed in a wall of the catheter.

17. The catheter assembly of claim 14, wherein the one or more side openings are in communication with at least one lumen in the catheter.

18. The catheter assembly of claim 1, wherein said catheter has a distal end that is not cut off.

19. The catheter assembly of claim 1, in combination with a shunt valve and distal catheter assembly.

20. The combination of claim 19, wherein the distal catheter assembly includes a distal catheter disposed at least partially in a distal sheath permanently attached below the patient's skin.

21. A method of installing a catheter assembly in a brain of a patient, the method comprising:
installing a sheath into the brain of the patient to create a permanent passageway between a surface of the brain and a ventricle within the brain;
removably inserting a catheter into the ventricle via the sheath.

22. The method of claim 21, further comprising, before the step of inserting the catheter, attaching an inner connection portion to a proximal end of the sheath, the inner connection portion arranged to be permanently attached to the brain; wherein the step of removably inserting a catheter includes removably inserting a catheter having an outer connection portion attached to a proximal end of the catheter.

23. The method of claim 22, further comprising connecting the inner and outer connection portions together.

24. A method of replacing an obstructed catheter of the catheter assembly of claim 1, the method comprising:
slidably removing the obstructed catheter from the sheath; and
slidably inserting a second catheter into a ventricle of the brain via the sheath.

25. The method of claim 25, wherein the step of slidably removing the obstructed catheter includes slidably removing the obstructed catheter from the sheath while the sheath remains attached to the brain.

26. A method of using the catheter assembly of claim 1, the method comprising withdrawing fluid from a ventricle within the brain.

27. The method of claim 26, wherein the step of withdrawing fluid includes withdrawing fluid via one or more lumens in the catheter.

28. The method of claim 26, wherein the step of withdrawing fluid includes withdrawing fluid via one or more openings disposed in a wall of the catheter.

29. The method of claim 26, wherein the step of withdrawing fluid includes withdrawing fluid via a space between an inner surface of the sheath and an outer surface of the catheter.

30. The method of claim 26, further comprising transferring the fluid from the ventricle to an abdominal cavity via a distal catheter.

31. The method of claim 26, wherein the step of transferring the fluid from the ventricle includes transferring the fluid via a distal catheter slidaby disposed in a distal sheath permanently attached to the patient.

32. A shunt comprising:
a catheter assembly arranged to be installed in a patient's brain;
a shunt valve; and
a distal catheter assembly, the distal catheter assembly including a distal sheath and a distal catheter slidingly disposed in the distal sheath;
wherein the distal sheath is arranged to be permanently subcutaneously attached to at least one of a neck, a chest and an abdomen of the patient.

33. The shut of claim 32, wherein a distal end of the distal catheter extends outwardly beyond a distal end of the distal sheath.

34. The shunt of claim 32, wherein the distal catheter fits snugly within the distal sheath.

35. The shunt of claim 32, wherein the catheter assembly includes a sheath arranged to be permanently attached to a portion of a brain and a catheter slidably disposed in the sheath.

36. A shunt comprising:
a catheter assembly including a sheath arranged to be permanently attached to a portion of a brain and a catheter slidably disposed in the sheath;
a shunt valve; and
a distal catheter.

37. The shunt of claim 36, wherein the catheter fits snugly within the sheath.

38. The shunt of claim 36, further comprising a space between an inside surface of the shunt and an outside surface of the catheter.

39. The shunt of claim 36, wherein the catheter and sheath are connected to the shunt valve.

40. The shunt of claim 36, further comprising a connector arranged to connect the sheath and catheter to the shunt valve.
